(19) United States
(12) Patent Application Publication
(10) Pub. No.: US 2013/0066205 A1
(43) Pub. Date: Mar. 14, 2013

(54) Ultrasound bone imaging assembly

(71) Applicants: Zvi Laster, Poriya Elite (IL); Ilan Weissberg, Givat Shmuel (IL); Adi Eldar, Kiryat Ono (IL); Avraham Levy, Herzeliya (IL); David Elkaim, Tel Mond (IL)

(72) Inventors: Zvi Laster, Poriya Elite (IL); Ilan Weissberg, Givat Shmuel (IL); Adi Eldar, Kiryat Ono (IL); Avraham Levy, Herzeliya (IL); David Elkaim, Tel Mond (IL)

(21) Appl. No.: 13/137,786

(22) Filed: Sep. 13, 2011

(51) Int. Cl. A61B 8/08

(52) U.S. Cl. 600/439

(57) ABSTRACT

A device for orienting a bone cutting tool with respect to a locking screw hole of an intramedullary nail inserted within a bone is provided. The device includes a device body which is configured with a cutting path for a cutting device and a distal end portion which is adapted for positioning against a surface of the bone. The device also includes an ultrasound probe holder which serves for aligning the cutting path with the screw locking feature using at least one ultrasound signal of at least one ultrasound probe attached to the device.

### FIELD OF THE INVENTION

The present invention generally relates to ultrasound bone imaging devices and, more particularly, but not exclusively, to an ultrasound bone imaging assembly for use in conjunction with fracture fixation.

### BACKGROUND OF THE INVENTION

Fracture reduction of a long bone may require alignment and reduction of the fracture using X-ray transillumination comprising, for example a C-arm X-ray system. A C-arm X-ray includes an X-ray source configured to be positioned on one side of a body portion of a subject and a detector configured to be positioned on a second side of the body portion. The C-arm is swiveled around the body portion in multiple axes to produce transillumination X-ray images.

In a fractured long bone, for example the femur, fracture fixation may require using an intramedullary (IM) rod, alternatively referred to as an IM nail. In IM rod fixation of the femur, C-arm X-ray transillumination may be used to image the fractured bone portions during reduction and in guiding the IM nail through the medulla of the fractured long bone to maintain alignment.

To prevent torsion and/or misalignment between the fractured bone portions, some IM rods include locking holes through which locking screws are placed.

To place the locking screws properly, in many instances an IM jig, having drill guides corresponding to the locking holes in the implanted IM rod, is externally aligned with the internally implanted IM rod, and secured in place.

The skin is incised at a given drill guide and a bore is drilled using a drill guide in the IM jig to guide the drill bit through the locking hole, thereby creating a bore through the bone and locking hole of an appropriate diameter to receive a locking screw.

The locking screw is then screwed through the locking hole and positioned so that screw shaft extends on either side of the IM rod. One or two locking screws are typically placed in the proximal portion of the IM rod as well as the distal portion of the IM rod, thereby preventing the above-noted torsion and/or misalignment between the fractured bone portions.

In spite of the use of an IM jig to locate and drill the locking holes through the IM nail, there can be alignment problems that cause delays in securing the locking screws in place. For example, deviation may occur between the position of the locking-hole on the (internally) implanted IM nail and the (external) drill guide on IM jig. The cause of this deviation may be a curvature that forms in the intramedullary nail due to a curvature in the long bone through which the IM rod is placed.

In light of the above-noted deviation and to ensure proper placement of locking screws, the above-noted C-arm X-ray transillumination is often used to locate the position of each locking hole and/or properly aim the locking screw into the locking hole.

The resulting extended use of the C-arm increases the exposure time of the patient to harmful ionizing radiation which can jeopardize patient health.

Additionally, X-ray transillumination presents a health risk to the physician, whose hands must function under the X-ray beam during fracture alignment, reduction, IM nail placement, drilling locking holes, and placement of locking screws. The many X-ray transillumination operations that a physician may perform throughout a lifetime collectively accumulate adverse ionizing radiation effects, putting the physician at a much higher risk than each individual patient.

Further, C-arm X-ray assemblies cannot be sterilized, thereby creating difficulty in maintaining the necessary sterile field during fracture reduction surgery.

In addition, a C-arm X-ray is a large, expensive assembly and, as a result, only one C-arm X-ray assembly exists in a small rural hospital, making it impossible to rapidly attend to multiple injured patients, for example following a local multiple vehicle road accident.

Using ultrasound to image a bone fracture are taught in:
i) Hacihaliloglu et al; “Bone Segmentation and Fracture Detection in Ultrasound Using 3D Local Phase Features”; Proceedings of the 11th international conference on Medical Image Computing and Computer-Assisted Intervention—MICCAI 2008; Vol. 5241/2008 pages 287-295; and
ii) Mahaisavariya et al “Technique of Closed Unlocked Femoral Nailing”; Injury; Volume 37, Issue 10, (2006) Pages 1000-1003.

The contents of all of the above documents are incorporated by reference as if fully set forth herein.

### SUMMARY OF THE INVENTION

According to an aspect of some embodiments of the present invention there is provided a method for reducing a fracture and detecting a locking hole of an intramedullary nail inserted within a medullary cavity.

The method includes the steps of scanning an ultrasonic wave signal in a direction perpendicular to an axis of the bone, ultrasonically detecting a fracture based on an echo of the ultrasonic wave signal reflected from the bone, reducing the fracture, and confirming fracture reduction using the ultrasonic wave signal.

The method further includes the steps of introducing an intramedullary nail having locking screw holes through the medulla of the bone, to span the fracture, slidably supporting an ultrasonic probe at a location of a locking screw hole of the intramedullary nail, and detecting the locking screw hole based on shape of the echo of the ultrasonic wave signal reflected from the intramedullary nail.

In some embodiments of the present invention the method includes drilling a bore through the bone and through the locking screw hole.

In some embodiments of the present invention the method includes rotating a locking screw through the bone and through the locking screw hole.

According to another aspect of some embodiments of the present invention there is provided an assembly for detecting the orientation and position of a locking screw hole of an intramedullary nail positioned within a medullary canal of a bone.

The assembly includes an ultrasonic transducer assembly having one or more transducers arranged to emit and detect ultrasonic signals, and an ultrasound imager operatively associated with the one or more transducers.

The ultrasound imager is configured for providing one or more images that indicate that the one or more transducers are at least one of: perpendicular to an intramedullary nail axis; and centered over a locking hole; of an intramedullary nail positioned within a medullary canal of a bone.

In of some embodiments of the present invention the ultrasound imager is additionally configured to provide one or more images that indicate at least one of: an orientation, a position, and drill bit image, during drilling of the locking screw hole of the intramedullary nail.

In of some embodiments of the present invention the assembly includes at least one elongate positioning bar, and at least one bone connector extending from the elongate positioning bar.

The connector is configured to connect the elongate positioning bar to at least one portion of a bone of a human subject.

The assembly further includes one or more ultrasound slidable transducer guides configured to slide along the at least one elongate positioning bar during the emission and detection of the ultrasonic signals.

In of some embodiments of the present invention the assembly includes at least one elongate positioning bar slidably mounted on at least one curved member having at least one bone connector configured to connect to at least one portion of a bone of a human subject.

In some embodiments of the present invention the at least one curved member includes at least two curved members, a first curved member configured to connect to a first portion, and a second curved member configured to connect to a second portion, of the human subject.

In some embodiments of the present invention the assembly includes an intramedullary rod connector configured to connect to the intramedullary rod in vivo and extend ex vivo.

The assembly further includes an elongate measuring bar configured to connect to the intramedullary rod connector, the elongate measuring bar including markings corresponding to the position of at least one locking screw hole, and an ultrasound transducer guide including the one or more transducers arranged to emit and detect ultrasonic signals and including a central bore configured for receiving a drill bit for drilling the bone through the at least one locking screw hole.

According to a further aspect of some embodiments of the present invention there is provided an assembly for drilling a bore through a locking screw hole of an intramedullary nail inserted within a medullary cavity of a bone.

The assembly includes a central channel configured for receiving a drill bit for drilling the bone through at least one locking screw hole in an intramedullary nail inserted within a medullary cavity of a bone, and one or more ultrasound transducers that emit and detect ultrasonic signals, the one or more ultrasound transducers being arranged around the central channel.

In of some embodiments of the present invention the one or more ultrasound transducers are operatively associated with an ultrasound beam imager configured for determining that one or more ultrasonic signals emitted and detected by the one or more ultrasonic transducers are at least one of: perpendicular to an axis of the intramedullary nail, and centered over a locking hole.

Thus, the present invention provides a method and assembly for facilitating reduction of bone fractures using ultrasound imaging as well as locking the distal holes of an intramedullary nail.

Although the use of ultrasound for identifying and reducing bone fragments provides numerous advantages over prior art approaches, locking the distal holes of an intramedullary nail is considered by many surgeons as the most demanding phase of intramedullary nailing of long bones.

This stage requires very accurate positioning of the C-arm, perpendicular to the distal nail longitudinal axis, until a perfect circle (representing the hole) is seen. Once the hole in the nail is identified, the surgeon must be accurate and steady in drilling through a convex surface of the femur, a task which can be difficult to achieve especially for less experienced surgeons. As a result, surgeons oftentimes repeat the drilling step thereby enlarging the bone hole and potentially weakening the bone.

While reducing the present invention to practice, the present inventors have devised an approach for setting a bone cutting path of a cutting device such as a drill using an ultrasound signal. Although the prior art describes use of an ultrasound probe for identifying the hole of an intramedullary nail [Yoshihisa et al. “Localization Detection of Transverse Holes in Intramedullary Nail Inserted into Femur with Flesh Using Ultrasound”, Nihon Kikai Gakkai Tokai Shibu Chiku Koenkai Koen Ronbunshu (2001)], such an approach utilizes a jig which supports an external ultrasound probe that is positioned against the skin and thus transmits an ultrasound signal through soft tissue (skin, fat, muscle) and hard tissue (bone) to image the nail. Due to the relatively long path that the ultrasound wave passes up to the bone and back (twice the thickness of the thigh, more than 10 cm) the ultrasound signal is severely attenuated in the soft tissue; in addition, the density differences between the soft tissues and the bone causes most of the ultrasound energy to be reflected back and not penetrating the bone to reach the IM nail at all. Thus in order to be able to image the IM nail (which is inside the bone) using external transducer, a high power ultrasound signal is needed, and this causes a patient safety issue as it can potentially damage surrounding tissues (heating and causing cavitation).

The present invention utilizes a device which is configured for:
(i) aligning a cutting path for a cutting device using an ultrasound probe; and (ii) positioning the ultrasound probe in a manner which enables bone and nail imaging without having to transmit the ultrasound signal through soft tissues.
   Thus, according to another aspect of the present invention there is provided a device for setting a cutting path which is aligned with a locking feature (e.g. hole) of an intramedullary nail. The device of the present invention includes a device body having a cutting path for a cutting tool and a holder for at least one ultrasound probe. As used herein, the term “cutting” when made in reference to bone refers to drilling, boring and the like using any suitable device, including, but not limited to, rotary cutters (e.g. drills), energy-based cutters (e.g. lasers) and the like. Several configurations of the present device are contemplated in the following section. The common denominators for all configurations is that for the task of locating the hole in the IM nail, the transducers are located either on top of the bone looking via the bone for the IM nail, or inside the nail hole looking via the bone outside for the drill, but in both cases the ultrasound wave doesn't pass via soft tissue of the thigh (or shin or forearm). Some of the configurations utilize a jig that is attached to the bone, stabilizes the transducers and enable locking them in place, while other configurations utilize hand held trocar that contains the transducers for navigation in a standard free hand technique.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only, and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware, or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system.

In an exemplary embodiment of the invention, one or more tasks according to embodiments of method and/or system as described herein may be performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1-3 show flow charts for fracture reduction and fixation using ultrasound, according to some embodiments of the invention;

FIGS. 4-5 show an ultrasound assembly for fracture reduction and fixation, according to some embodiments of the invention;

FIG. 6 shows an alternative embodiment for fracture fixation shown in FIGS. 4-5, according to some embodiments of the invention;

FIG. 7-8 show ultrasound hand held assemblies for fracture reduction and fixation, according to some embodiments of the invention;

FIGS. 9-10 show details of the handheld assemblies shown in FIG. 8, according to some embodiments of the invention; and

FIG. 11 shows another option for the hand held assembly, alternative to FIG. 8.

### DETAILED DESCRIPTION OF EMBODIMENTS OF THE INVENTION

The present invention generally relates to ultrasound bone imaging assemblies and, more particularly, but not exclusively, to an ultrasound bone imaging assembly for use in conjunction with a fracture fixation system.

Before explaining at least one embodiment of the invention in detail, it is to be understood that the invention is not necessarily limited in its application to the details of construction and the arrangement of the components and/or methods set forth in the following description and/or illustrated in the drawings. The invention is capable of other embodiments or of being practiced or carried out in various ways.

Referring now to the drawings:

### Ultrasound Fracture Reduction

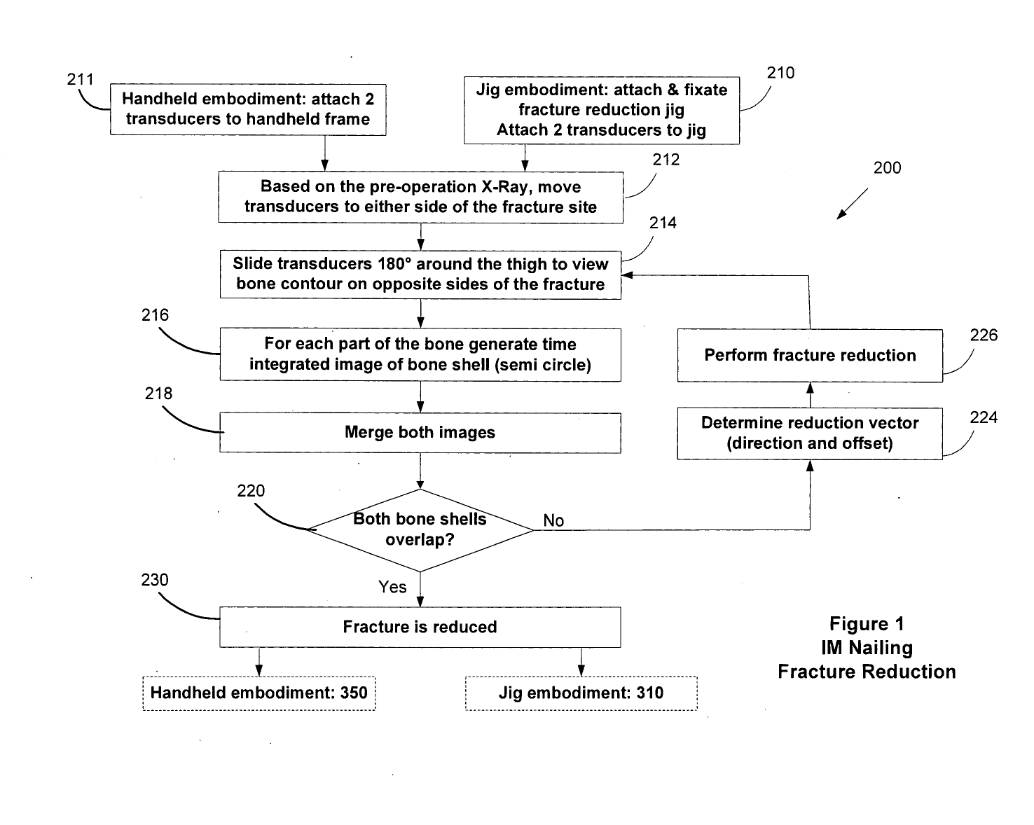

FIG. 1 shows an intramedullary nailing flow chart 200 wherein at a stage 210 a fracture reduction jig is attached proximate to the fractured bone and two fracture ultrasound transducers are placed in sliding jig holes.

Alternatively, at a stage 211, when using a handheld fracture unit, two transducers are placed in the transducer frame.

At a stage 212, based on the preoperative x-ray, the transducers are moved to either side of the fracture site.

At a stage 214 the two transducers, whether in a jig-based system or in a handheld system, are moved 180° around the thigh to view contour of the fracture fragments.

At a stage 216, each portion of the fracture is visualized using the ultrasound images produced by the transducers, with the image being constructed using integration of data from different projections that is known in the art. The constructed images are merged at a stage 218, and produce two semicircles, corresponding to the edges of the bone shell (cortex) of the fracture fragments.

Overlap in the two semicircles of the bone shell images signifies that the fracture has been properly reduced at a stage 230.

At a stage 220 the image is analyzed in two dimensions and it is determined if there is overlap in the bone fragments, signifying the above-noted fracture reduction.

Alternatively, if the above-noted parameters of stage 220 are not adequate, a stage 224 is accessed to determine the required vector for reduction of the fracture. The fracture is aligned and reduced at a stage 226. Following stage 226 transducers are again utilized in stage 214 through stage 220 to ensure that the fracture has been reduced at stage 230.

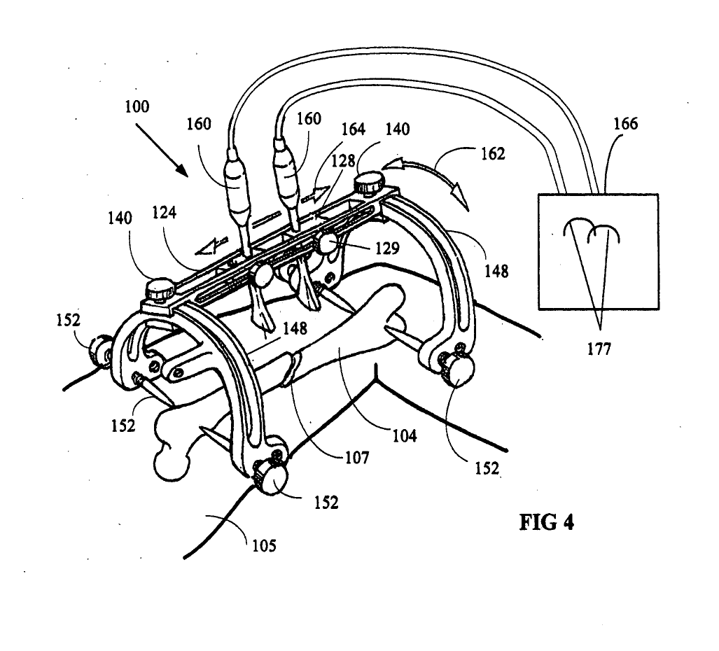

FIG. 4 shows a fracture jig 100 used in this figure for fracture reduction. The jig has positioning spikes 152 that pass through thigh tissue 105 and secure into a femur 104, alternatively referred to as thigh bone 104.

Ultrasound transducers for fracture reduction 160 are placed into transducer bases 128 along a longitudinal rail 124 on either side of a fracture 107. To determine the above-noted bone contour and fracture alignment, the fracture reduction transducers 160 are moved in directions 164 along longitudinal rail 124 and positioned above the two bone fragments on either side of the fracture, as close as possible to the fracture.

Once fracture reduction transducers 160 are in the correct position, transducer bases 128 are locked in position with transducer locks 129, and longitudinal rail 124 is rotated in directions 162 along semicircular rails 148 after releasing rail stops 140. Based on the accumulated transducers' data during rotation in directions 162, an integrated echo image is generated on imaging display 166.

At this stage the surgeon manipulates femur 104 in order to align fracture 107 in preparation for driving an intramedullary nail through femur 104.

Alignment of fracture 107 is confirmed on an imaging display 166, when two shells 177, alternatively two semi circles 177, representing the proximate bone cortex of the two bone fragments, appear partially aligned and/or overlapping.

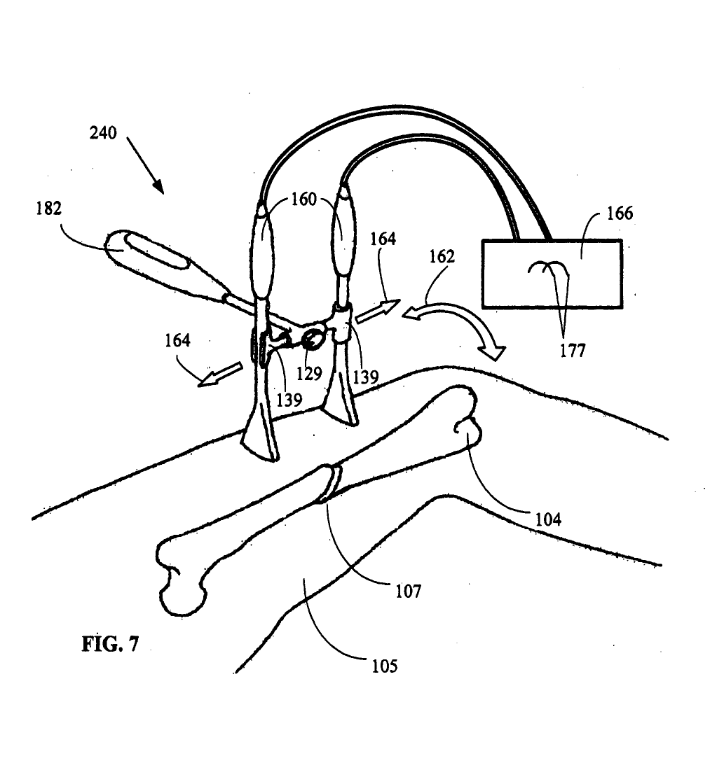

FIG. 7 shows a handheld ultrasound assembly 240 for fracture reduction in which fracture reduction transducers 160 are placed into horizontal transducer holders 139 and placed proximate to fracture 107. Horizontal transducer holders 139 are moved laterally in directions 164, using a handle 182, to the optimal position on either side of fracture 107 and rotated in direction 162 to define the above-noted bone contour. Fracture 107 is manipulated as noted above, until semi-circles 177 partially overlap and/or align as noted above.

### Ultrasound Locating of the IM Nail Locking Holes and Drilling

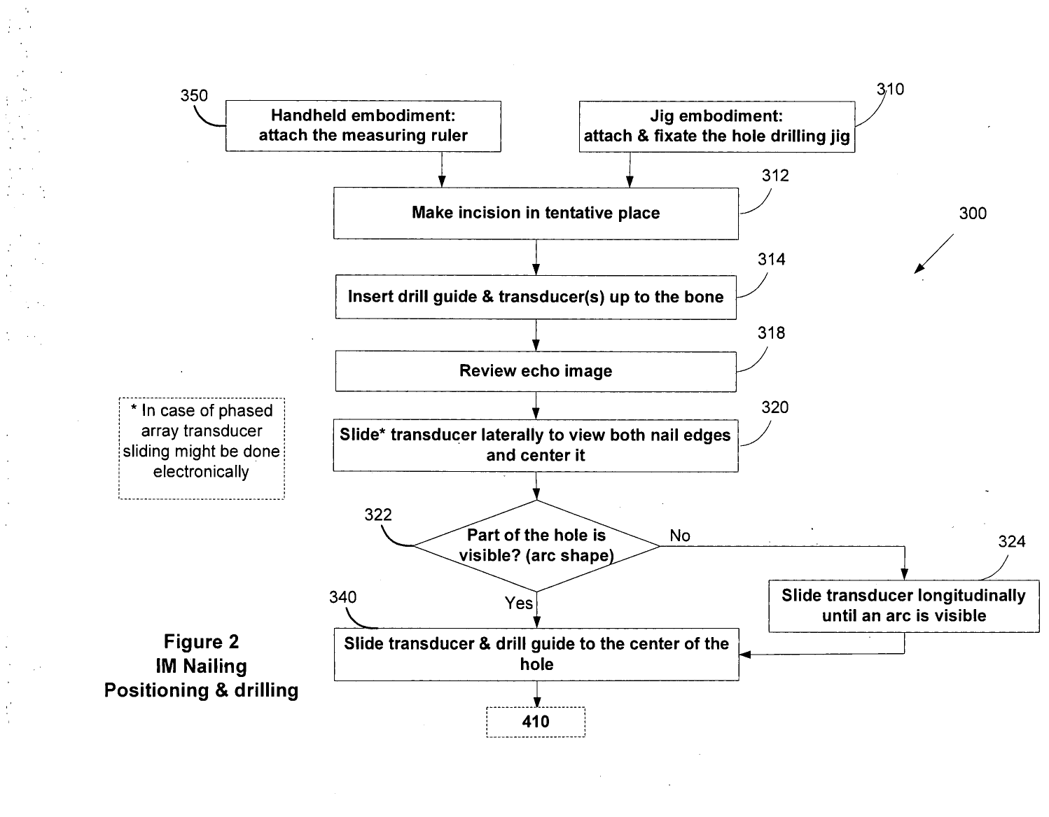

Following fracture reduction, a stage 310 is accessed, as seen in FIG. 2, in which a drill guide is attached to the fracture jig, in a drilling alignment hole corresponding to a locking hole in the IM nail.

Alternatively, in handheld fracture reduction, a stage 350 is accessed and a ruler is used to mark the incision area into which the locking screw is to be placed.

At a stage 312 an incision is made where the bore for the screw will be drilled. Whether using handheld or jig-based techniques, a drill guide equipped with one or more ultrasound transducers, is pressed up against the bone in a stage 314 through the incision made at stage 312.

The ultrasound echo image is reviewed in a stage 318 in order to ensure precise alignment and/or angulation between the drill guide and the locking hole of the IM nail.

From review of the echo image at stage 318, whether using a handheld transducer frame or a complete drilling jig, a stage 320 is accessed and the transducers are slid laterally to view the nail edge and/or hole image.

At a stage 322 the echo image is reviewed and checked to determine if part of the screw hole is visible in the form of an arc shape.

If part of the screw hole is visible, at stage 340 the transducer and drill guide are moved until aligned with the center of the hole.

Alternatively, if at stage 322 the echo image does not display any part of the screw hole, for example an arc shape is not visible, misalignment is indicated; and a stage 324 is accessed.

At stage 324, the transducers are slid longitudinally along with the hole-drilling guide until such time that a portion or all of a locking hole is visible and aligned.

For the fracture jig embodiment, it should be noted that sliding may be performed either manually or using a power source comprising, inter alia, a motor, a magnet propelled positioning palette, or a pressurized hydraulic system. The many options for sliding transducers along an IM positioning jig are well known to those familiar with the art. It should also be noted that the present invention can be used in conjunction with a is phased array transducers.

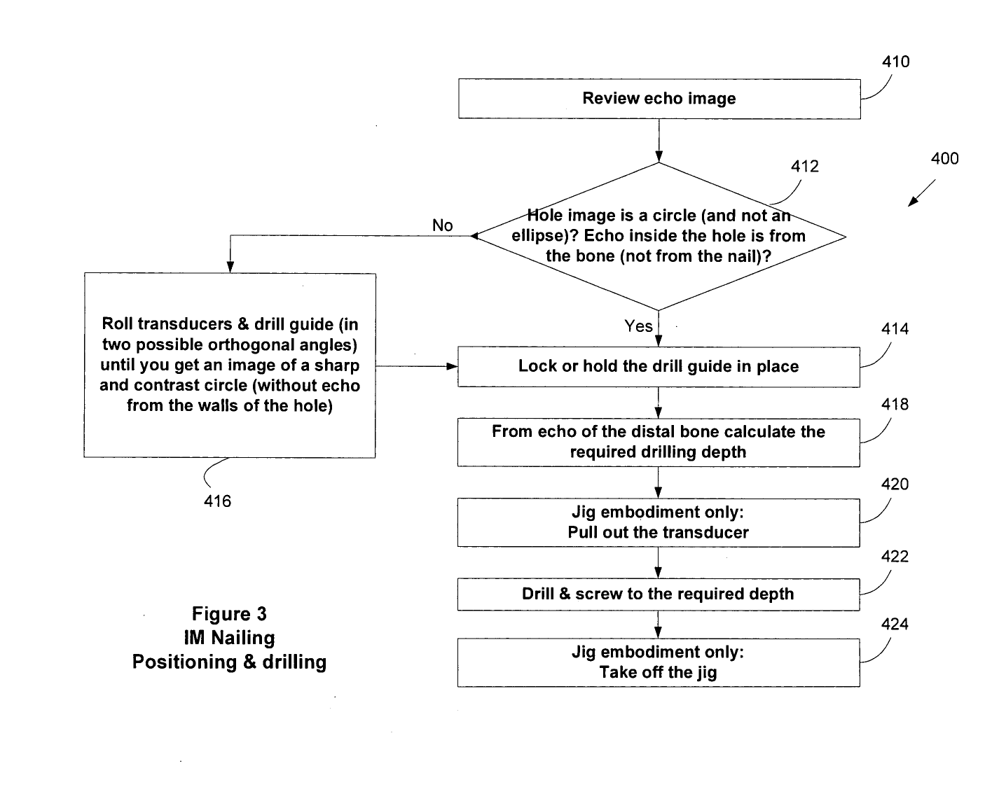

Once it is determined at stage 340 that the drill guide is centered over the locking hole of the intramedullary rod, a drilling process 400 is accessed as seen in FIG. 3.

A stage 410 is initially accessed, wherein the echo ultrasound image is reviewed. At a stage 412, it is determined whether the hole is a circle or an ellipse.

Additionally, the ultrasound echo is analyzed to determine if the echo is from the distant bone or from the walls of the nail hole.

For example, a circle, or ellipse, in the ultrasound image is generated by the edges of the locking hole. If alignment is correct, an ultrasound echo is received from the distant bone cortex through the locking hole. In such cases, the analyzed ultrasound echo will demonstrate signal delay consistent with the distance to the distant bone cortex.

If the transducer and/or drill guide alignment is incorrect, the received ultrasound echo will be generated from one or more portions of the vertical walls of the nail hole.

The determination of potential drilling misalignment occurs when the analyzed ultrasound echo demonstrates a short signal delay that signifies that the ultrasound echo is being reflected from a proximate structure, rather than the distant bone cortex.

When the ultrasound image displays a single circle of the locking hole with peak sharpness, (between the echo from the distant bone and the edges of the locking hole) a stage 414 as accessed. At stage 414, the locking of the drill guide in position over the locking hole, takes place. Alternatively, in handheld embodiments, the drill guide is held in place with the operator's hand.

Should stage 412 show that the hole is inordinately elliptical and/or that part of the echo is from the proximate wall of the nail hole, a stage 416 is accessed, wherein the transducer and drill guide are rolled, possibly in two orthogonal angles, until the image produced by the ultrasound is sharp, and shows a circle with an ultrasound echo from the distant bone, as opposed to an ultrasound echo from the walls of the nail locking hole.

Once the image is determined to have correct shape, stage 414 is accessed and, as noted above, the drill guide is locked (or held) in place. At a stage 418, the echo of the ultrasound is used to calculate the required drilling depth.

At a stage 420, when using a fracture jig, the transducer is removed from the drill guide. At a stage 422, a drill bit is placed in the drill guide and the bore is created to the required depth through the bone.

Following drilling of the bone through the locking hole and rotating the screw to the required depth, drilling process 400 is repeated until all locking screws have been drilled into place.

Following completion of all drilling and placement of locking screws, when using a fracture jig, at a stage 424 the jig is removed from the bone.

It will be appreciated that use of the present drill guide provides two advantages: it enables correct alignment between the proposed cutting path through the bone and the hole in the nail while it also stabilizes the cutting tool throughout the bone cutting procedure thereby preventing the cutting tool from slipping off the convex bone surface or straying during cutting.

There are a variety of assemblies that can accomplish the above-noted task of locating the locking screw hole, drilling and screw placement, of which but a few will be herein described.

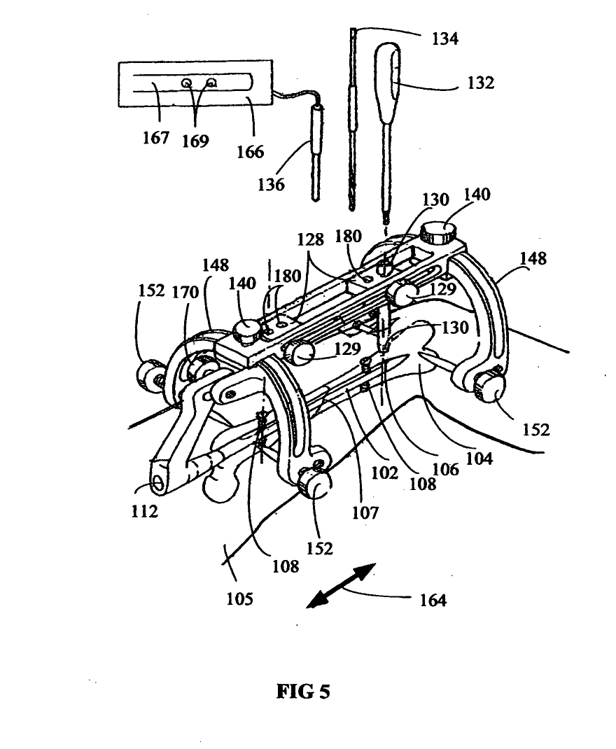

FIG. 5 shows an intramedullary nail 102 having been passed through the intramedullary canal. An intramedullary nail connector 112 has been attached to intramedullary nail 102 and secured with a connector trimmer to jig 100. Initially ultrasound transducers 136 are placed through a guide sleeve 130 in guide holes 180 in transducer bases 128. Transducer base(s) 128 is moved in directions 164 and transducer 136 is used to locate a locking hole 106 in intramedullary nail 102.

Upon locating locking hole 106, display 166 shows an IM rod image 167 with symmetrical IM locking hole 169 with the highest contrast corresponding to one of the locking holes 106.

Upon locating locking hole 106, transducer 136 is removed from guide sleeves 130 and replaced with a drill bit 134 to drill a hole across femur 104 through locking hole 106.

A locking screw 108 is then screwed into femur 104 via guide sleeve 130 through locking hole 106. Following introduction of proximal and distal locking screws 108, spikes 152 are removed from femur 104 and jig 100 is removed from thigh 105.

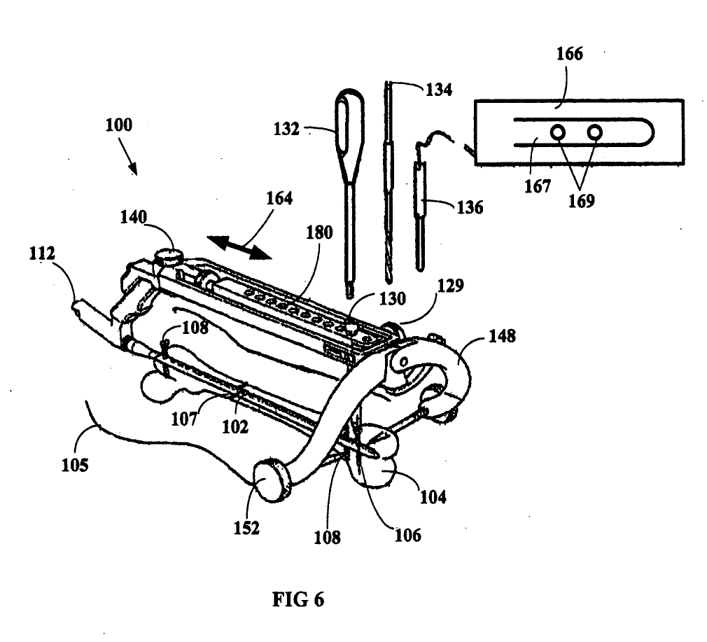

FIG. 6 shows an alternative embodiment of the jig assembly 100 that attaches directly to intramedullary nail 102 with connector 112 and only one set of positioning spikes 152 attached to the distal portion of femur 104.

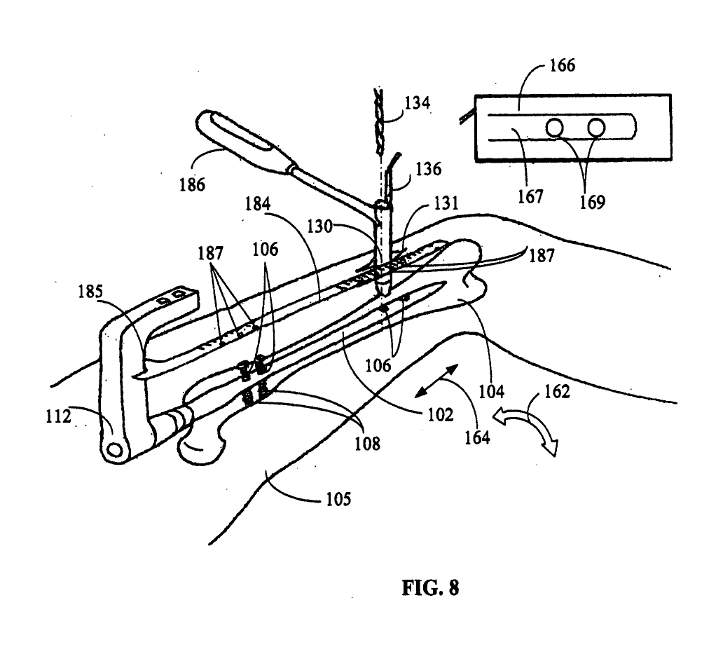

FIG. 8 shows a handheld ultrasound assembly for locating the IM nail holes and drilling. An IM nail 102 attached to intramedullary nail connector 112 and passed through femur 104 using standard surgical IM nailing technique.

A measuring rod 184, alternatively flexible measuring tape 184, is placed so that a connector end 185 is in contact with, and/or connected to intramedullary nail connector 112 and positioned parallel to IM nail 102.

Measuring rod 184 includes markings 187 corresponding to the position of locking holes 106 and thigh 105 is incised to form an incision 131 at the approximate position of each locking hole, optionally following marking thigh 105 with a skin marker.

Guide sleeve 130 on a handle 186 is placed into incision 131 to contact femur 104 and swiveled along the X and Y axes in directions 164 and 162 until a symmetrical locking hole 169 with the highest contrast is visualized on imaging display 166.

Drill bit 134 is then passed through drill guide to bore across femur 104 through locking hole 106, following which screw 108 is rotated into place through locking hole 106.

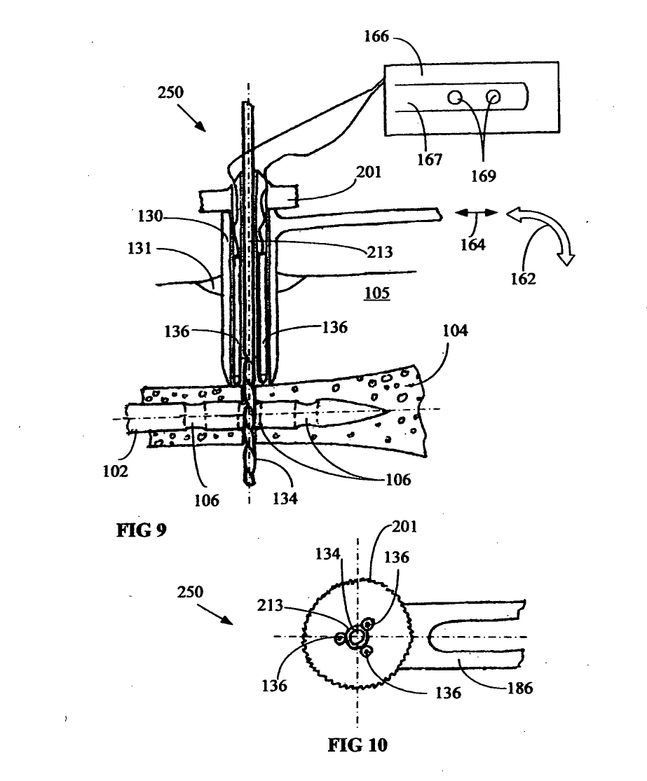

FIG. 9 shows details of an integrated drill guide assembly 250, in cross section, passing through a cross section of femur 104 and IM nail; and FIG. 10 shows an aerial view of integrated drill guide assembly 250. Integrated drill guide assembly 250 incorporates three ultrasound transducers 136 in a transducer jacket assembly 201 that slides within guide sleeve 130.

As is mentioned hereinabove, guide assembly 250 is configured such that when a distal end portion thereof is positioned against the bone, transducers 136 contact the surface of the imaged bone directly or through an ultrasound conductive material (e.g. ultrasound gel). It will be appreciated that configurations in which transducers 136 are separated from the bone (by millimeters to centimeters) are also envisaged herein. In such cases, an ultrasound conductive material (e.g. ultrasound gel) is used to provide a path for the ultrasound signal. One example of such a configuration can include transducer 136 disposed within sleeve 130 and displaced (several cm) from the surface of the imaged bone via an ultrasound gel.

Prior to drilling femur 104, transducer jacket assembly 201 and guide sleeve 130 are passed, through incision 131 and against femur 104, proximate to locking hole 106. Guide sleeve 130 is manipulated in the above-noted manner in directions 162 and 164, until locking hole 106 is imaged on the above-noted ultrasound imager. Transducer jacket assembly 201 includes a central drill bore 213 through which drill bit 134 is passed, and femur 104 is drilled in the above-noted manner. During drilling, an ultrasound image is optionally used to image drill bit 134 during passage through femur 104. Optionally, guide sleeve 130 is additionally configured to receive and guide screw 108 following drilling and the ultrasound image is used to image screw 108 during passage through femur 104.

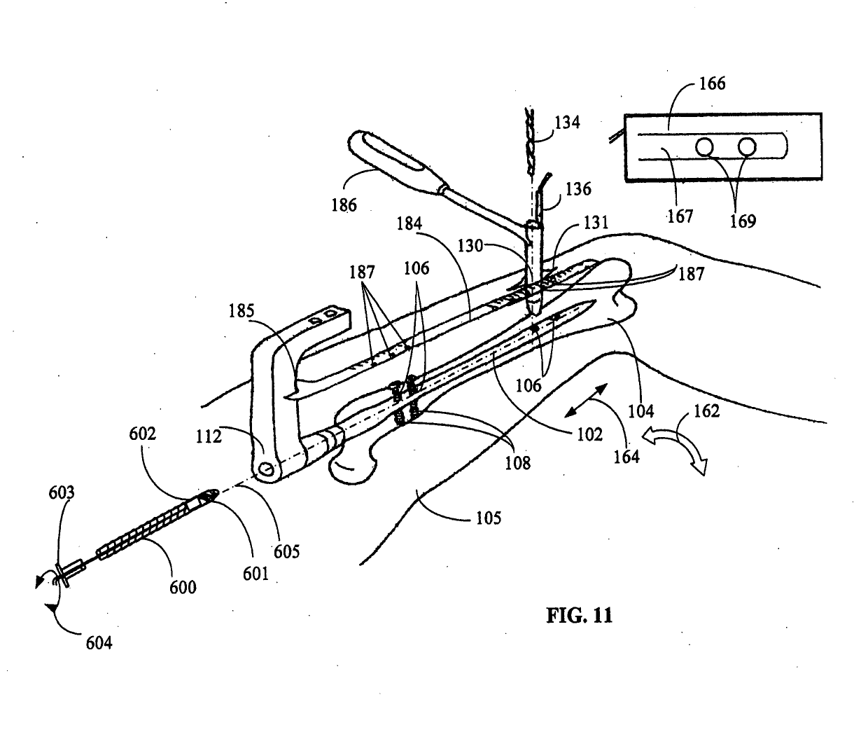

FIG. 11 is similar to FIG. 8, where the only difference is the usage of a different type of transducer 136. In this configuration the transducer is composed of two distinct parts: a transmitter and a receiver (a.k.a. a transducer pair). The transmitter part 136 is positioned in the guide sleeve 130 as in FIG. 8, while the receiver part 601 is inserted into a cannulated IM nail 102 using an insertion device 600. The insertion device 600 is comprised from a rigid or flexible wire, with a handle 603 on one end and the receiver 601 on the other end. The receiver 601 should be inserted into the cannulated IM rod 102 along axis 605 such that its position would be in the middle of the required locking hole 106. As we know a-priori the precise dimensions of the IM nail 102, accurate positioning and orientation of the receiver 601 can be achieved by longitudinal and angular measuring marks on the wire 600 that can be aligned with a reference mark on the nail connector 112. The receiver active surface 602 should be perpendicular to the locking hole 106, this can be achieved by rotating the device 600 in directions 604 so the angular mark on the wire 600 is aligned with the reference mark. Additional option, referring to the same FIG. 11, is that part 601 comprised of the full transducer (transmitter & receiver) and in this configuration the previous transmitter part 136 is not needed at all. In this case the transducer 601 will transmit the ultrasound wave from the IM nail hole, in a perpendicular direction, via the bone and it could image the drill once the drill is on top of the bone in the correct location.

Notes: a) The procedure of insertion the receiver 601 to the IM nail 102 is applicable only to a cannulated nail.

b) The procedure can be done either before or after the IM nail is inserted into the bone 104.

It will be appreciated that although the above description relates to intrameduallry nail locking in a femur, treatment of other long bones (e.g. tibia) can also be effected using the present device and method.

It is expected that during the life of a patent maturing from this application many relevant Ultrasound bone alignment assemblies will be developed and the scope of the term “Ultrasound bone alignment assembly” is intended to include all such new technologies a priori.

As used herein the term “about” refers to ±10%.

The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical, and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications, and variations will be apparent to those skilled in the art. Accordingly, it is intended to embrace all such alternatives, modifications, and variations that fall within the spirit and broad scope of the appended claims.

All publications, patents, and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A method of reducing a fracture and detecting a locking hole of an intramedullary nail inserted within a medullary cavity, comprising the steps of:
a) scanning an ultrasonic wave signal in a direction perpendicular to an axis of the bone;
b) ultrasonically detecting a fracture based on an echo of said ultrasonic wave signal reflected from the bone;
c) reducing said fracture;
d) confirming fracture reduction using said ultrasonic wave signal;
e) introducing an intramedullary nail having locking screw holes through the medulla of the bone, to span said fracture;
f) slidably supporting an ultrasonic probe at a location of a locking screw hole of said intramedullary nail; and
g) detecting said locking screw hole based on shape of the echo of said ultrasonic wave signal reflected from said intramedullary nail.

2. The method according to claim 1, including:
h) drilling a bore through the bone and through said locking screw hole.

3. The method according to claim 2, including:
i) rotating a locking screw through said bone and through said locking screw hole.

4. An assembly for detecting the orientation and position of a locking screw hole of an intramedullary nail positioned within a medullary canal of a bone, the assembly comprising:
a) an ultrasonic transducer assembly having one or more transducers arranged to emit and detect ultrasonic signals; and
b) an ultrasound imager operatively associated with said one or more transducers, said ultrasound imager configured for providing one or more images that indicate that said one or more transducers are at least one of:
I) perpendicular to an intramedullary nail axis; and
II) centered over a locking hole,
of an intramedullary nail positioned within a medullary canal of a bone.

5. The assembly according to claim 4, wherein said ultrasound imager is additionally configured to provide one or more images that indicate at least one of:
I) an orientation;
II) a position; and
III) drill bit image,
during drilling of said locking screw hole of said intramedullary nail.

6. The assembly according to claim 4, including:
I) at least one elongate positioning bar;
II) at least one bone connector extending from said elongate positioning bar, said connector configured to connect said elongate positioning bar to at least one portion of a bone of a human subject; and
III) one or more ultrasound slidable transducer guides configured to slide along said at least one elongate positioning bar during said emission and detection of said ultrasonic signals.

7. The assembly according to claim 4, including: at least one elongate positioning bar slidably mounted on at least one curved member having at least one bone connector configured to connect to at least one portion of a bone of a human subject.

8. The assembly according to claim 7, wherein said at least one curved member comprises at least two curved members:
I) a first curved member configured to connect to a first portion of the human subject; and
II) a second curved member configured to connect to a second portion, of the human subject.

9. The assembly according to claim 4, including:
I) an intramedullary rod connector configured to connect to said intramedullary rod in vivo and extend ex vivo;
II) an elongate measuring bar configured to connect to said intramedullary rod connector, said elongate measuring bar including markings corresponding to the position of at least one locking screw hole; and
III) an ultrasound transducer guide including said one or more transducers arranged to emit and detect ultrasonic signals and including a central bore configured for receiving a drill bit for drilling the bone through said at least one locking screw hole.

10. An assembly for drilling a bore through a locking screw hole of an intramedullary nail inserted within a medullary cavity of a bone, the assembly comprising:
a) a central channel configured for receiving a drill bit for drilling the bone through at least one locking screw hole in an intramedullary nail inserted within a medullary cavity of a bone; and
b) one or more ultrasound transducers that emit and detect ultrasonic signals, said one or more ultrasound transducers being arranged around said central channel.

11. The assembly according to claim 10, wherein said one or more ultrasound transducers are operatively associated with an ultrasound beam imager configured for determining that one or more ultrasonic signals emitted and detected by said one or more ultrasonic transducers are at least one of:
i) perpendicular to an axis of said intramedullary nail; and
ii) centered over a locking hole.

12. A method of setting a bone cutting path in a bone fitted with an intramedullary nail comprising:
(a) positioning a guide having a cutting path for a cutting device against a surface of the bone;
(b) aligning at least one ultrasound probe with said guide; and
(c) orienting said cutting path of said guide with respect to a locking screw using an ultrasound signal from said at least one ultrasound probe.

13. The method of claim 12, wherein said ultrasound signal does not travel through soft tissue.

14. The method of claim 13, wherein said at least one ultrasound probe is positioned in direct contact with the surface bone.

15. The method of claim 12 further comprising (d) using said cutting device to cut into the bone along said cutting path of said guide.

16. The method of claim 13, wherein said cutting device is a drill.

17. The method of claim 12, wherein said guide is a hollow tube and said at least one ultrasound probe is attached to or fitted within said hollow tube.

18. The method of claim 12, wherein said at least one ultrasound probe is separated from said bone by ultrasound-conductive material.

19. The method of claim 17, wherein the lumen of said hollow tube forms said cutting path.

20. The method of claim 12, wherein (c) is effected by turning said intramedullary nail within an intramedullary space of the bone.

21. A device for orienting a bone cutting tool with respect to a locking screw hole of an intramedullary nail inserted within a bone comprising:
(a) a device body being configured with a cutting path for a cutting device and having a distal end portion adapted for positioning against a surface of the bone; and
(b) ultrasound probe holder for aligning said cutting path with said screw locking feature using at least one ultrasound signal of at least one ultrasound probe attached to the device body.

22. The device of claim 21, wherein said device is a hollow tube being designed for holding said at least one ultrasound probe in or around said hollow tube.

23. The device of claim 21, further comprising bone anchoring elements.

24. The device of claim 23, wherein said bone anchoring elements are disposed on or around said distal end portion.

25. The device of claim 21, wherein said ultrasound probe holder maintains said at least one ultrasound probe in contact with said surface of the bone when said distal end portion of said device body is positioned against said surface of the bone.

26. The method of claim 12, wherein the ultrasound probe is inside the screw hole of the intramedullary nail.

27. The method of claim 12, wherein the ultrasound probe is composed of distinct transmitter and receiver parts, wherein the transmitter part is aligned with the said guide and the receiver part is inside the screw hole of the intramedullary nail.

28. The device of claim 21, wherein the ultrasound probe holder is attached to the screw hole of the intramedullary nail.

29. The device of claim 21, comprising two ultrasound probe holders, such that the first one is attached to the device body and the second one is attached to the screw hole of the intramedullary nail.
